(19) United States
(12) Patent Application Publication
(10) Pub. No.: US 2022/0378534 A1
(43) Pub. Date: Dec. 1, 2022

(54) SURGICAL ROBOT

(71) Applicant: Olympus Winter & Ibe GmbH, Hamburg (DE)

(72) Inventors: Thorsten JUERGENS, Hamburg (DE); Andreas NOE, Hamburg (DE)

(73) Assignee: Olympus Winter & Ibe GmbH, Hamburg (DE)

(21) Appl. No.: 17/743,752

(22) Filed: May 13, 2022

Related U.S. Application Data
(60) Provisional application No. 63/192,632, filed on May 25, 2021.

(51) Int. Cl. A61B 34/37; G06F 3/01; G10L 15/22; A61B 90/00

(52) U.S. Cl. CPC A61B 34/37; G06F 3/013; G06F 3/015; G10L 15/22; A61B 90/37; G10L 2015/223

(57) ABSTRACT

A surgical robot system including: at least one slave manipulator for handling a surgical instrument within a body of a patient; and a control console, including: at least one master operating unit configured to be manually operated by an operating person to at least control the at least one slave manipulator; a display for displaying information; and at least one switch configured to: couple the at least one master operating unit with the at least one slave manipulator; decouple the at least one master operating unit from the at least one slave manipulator, and when the at least one master operating unit is decoupled from the at least one slave manipulator, couple the at least one master operating unit with the display.

### CROSS-REFERENCE TO RELATED APPLICATION

The present application is based upon and claims the benefit of priority from U.S. Provisional Application No. 63/192,632, filed on May 25, 2021, the entire contents of which is incorporated herein by reference.

### BACKGROUND

### Field

The present disclosure relates to a surgical robot and a method for operating the same.

### Prior Art

So-called robotic systems or surgical robots are used for minimally invasive surgery. Such systems or respectively robots have, for example, a manipulator with a surgical instrument, wherein the manipulator for treating a patient is controlled by an operating input unit and by a control command of the operating input unit. Usually, the manipulator is configured as a multi-jointed robot arm, wherein the surgical instrument or instruments are arranged on the end of the robot arm. In particular, the surgical robots have two manually operable operating units with which the advancement and angle of two robotic arms and the angling and the opening of the end effectors of a hand-held instrument being used are controlled.

For example, a manipulator system or respectively surgical robot for treating a person is known from DE 11 2016 003 481 T5.

### SUMMARY

An object is to expand the application possibilities of a surgical robot, wherein at the same time high requirements for the sterility of the robot are to be met in a simple manner.

Such object can be achieved by a surgical robot with at least one manipulator for handling a surgical instrument within a body of a patient and with a control console for controlling the surgical robot, wherein the control console has at least one operating unit, which can be manually operated by an operating person, for the at least one manipulator and a display field for displaying information, wherein the at least one operating unit is coupled with the at least one manipulator so that, when the at least one operating unit is actuated, the at least one manipulator is or becomes actuated, wherein the control console has an actuatable decoupling device and an actuatable coupling device, wherein the decoupling device is configured such that, when the decoupling device is actuated, the operating unit is or becomes decoupled from the manipulator, and wherein the coupling device is configured that, when the coupling device is actuated, the operating unit is or becomes coupled with the display field.

In one operating mode (coupling of the operating unit with the display field) an actuating or a controlling of the display field directly via the manually actuatable operating unit, which is provided in another operating mode (coupling of the operating unit with the manipulator), such as exclusively, for the control of the manipulator, can take place after the operating unit is decoupled from the manipulator. If, for example, the display field is configured as an operating panel, such as, for example, a touch panel or the like, it is not required for the operating person to operate the display field via other input devices during the use of the surgical robot, since, in this operating mode in which the operating unit is coupled with the display field, the display field can be controlled via the operating unit (for the manipulator), while at the same time the operating unit can be decoupled from the manipulator. In this way, touching the display field, for example, a touch panel, is avoided. In addition, it is not necessary to sterilize the surface of the display field or to apply a protective film to the display field.

The display field can also be configured as a video panel or the like for displaying an operation image.

In addition, the surgical robot can have one operating unit for each of the two hands of an operating person, which units can be each coupled with a manipulator in the working mode of the surgical robot for examining or operating on a person and can be controlled by the control commands of the operating units. The manipulator can be configured as a multi-jointed, robot arm. In this regard, a surgical instrument can be arranged at the end of a robot arm.

Because the operating units can be provided to control the manipulators in a first mode when the operating units are coupled with the manipulators (and decoupled from the display field) and the operating units can be provided in a second operating mode to control the display field or respectively elements on the display field after a decoupling from the manipulators and a coupling with the display field, it is not required to sterilize other input devices, such as, for example, control mice or the like, for the display field or to cover them with a sterile coating.

The surgical robot can have an endoscope with a camera endoscope or respectively a video endoscope, a video processor, a light source, an insufflator, an instrument for HF surgery (high-frequency surgery) and/or an ultrasonic surgery instrument and/or at least one light for illuminating an external operating field and/or a control for an operating table. In addition, the surgical robot can also have a video recorder or a video distributor for use with different video sources on multiple screens.

In one embodiment, the manipulator or manipulators can be configured as robotic arms, wherein the robotic arms have, for example, surgical instruments with and/without end effectors on their arm ends.

The manually operable operating units can be used alternatingly for the control of the manipulators (when the operating units are coupled with the manipulators and when the operating units are decoupled from the display field) or for the control of the display field of the control console (when the operating units are coupled with the display field and when the operating units are decoupled from the manipulators). To decouple, for example, the operating unit from the manipulator, it can be provided that, when the decoupling device is actuated manually by an operating person, a decoupling signal is generated so that the operating unit is decoupled from the manipulator by the decoupling signal. Accordingly, it can also be provided that, for the coupling of the operating unit with the display field, the coupling device is or becomes actuated so that a coupling signal is generated by the coupling device so that the operating unit is coupled with the display field by the coupling signal.

Moreover, when the operating unit is coupled with the display field, a display element on the display field can be activated by actuating the operating unit. This makes it possible, for example, to control the display field or respectively display elements on the display field by the operating unit.

The decoupling device can be configured as a pedal or as a button. Thus, the decoupling of the operating unit from the manipulator by an operating person can be possible via an actuatable foot pedal or by a manually actuatable button.

The pedal can be configured as a decoupling pedal in this case.

The decoupling device for decoupling the operating unit from the manipulator can have in general an operating element so that, when the operating element of the decoupling device is actuated, the operating unit is or becomes decoupled from the manipulator.

In addition, the coupling device can be configured as a button or switch. Here, the coupling device for coupling the operating unit with the display field can be configured as an operating element actuatable by an operating person so that, after the operating element, for example button or switch, is actuated, the operating unit is or becomes coupled with the display field.

The decoupling device and the coupling device can be configured in a button, such as a foot button, such that, at a first switching point of the button, the operating unit is coupled with the manipulator and the operating unit is decoupled from the display field and that, at a second switching point of the button, the operating unit is decoupled from the manipulator and the operating unit is coupled with the display field. This makes it possible, for example, that a decoupling between the manipulator, such as, for example, a robotic arm, and the operating unit takes place via a button on the operating unit, wherein, in a second operating mode, a coupling of the operating unit with the display field takes place via a second, such as subsequent, switching point on the button.

In one embodiment, the button can also be configured as a foot button or respectively pedal or the like.

Furthermore, before the decoupling device is actuated, the operating unit can be decoupled from the display field and/or that, during or after the actuation of the coupling device, the operating unit can be decoupled from the manipulator.

Furthermore, the actuation of the decoupling device and the actuation of the coupling device can be executed or are executed at the same time or temporally one after another.

Moreover, the coupling device can be configured with a gaze sensor device, wherein the gaze sensor device can be configured to couple the operating unit with the display field when it is detected or after it is detected that an alignment of a gaze of the operating person is oriented toward the display field. Here, the gaze sensor device can be arranged in the region of the display field and oriented toward the face of the operating person to detect the gaze of the operating person. It is thereby detected or respectively established whether the operating person is looking directly at the display field. Overall, this increases the operational safety during operation of the surgical robot.

In addition, the coupling device can be configured with a speech recognition device, wherein the speech recognition device can be configured to couple the operating unit with the display field during or after detection of a speech utterance, such as a speech command, of the operating person. For this purpose, the speech recognition device can have a microphone in order to detect, for example, a speech command of the operating person. After the analysis and detection of a corresponding speech command, the operating unit can be coupled with the display field.

In addition, the coupling device can be configured with an electromyography receiver for receiving electromyography signals of the operating person, wherein the electromyography receiver can be configured to couple the operating unit with the display field during or after detection of electromyography signals of the operating person. For this purpose, the electromyography receiver can have corresponding sensors with which corresponding electromyography signals are received or respectively detected, for example, on an extremity or the head of the operating person.

Furthermore, the coupling device can be configured with an electroencephalography receiver for receiving electroencephalography signals of the operating person, wherein the electroencephalography receiver can be configured to couple the operating unit with the display field during or after detection of encephalography signals of the operating person. The coupling between the operating unit and the display field, such as operating panel, thereby takes place after decoupling between the operating unit and the manipulator, such as robotic arm, has taken place after an evaluation of an electroencephalogram which will be or is received with corresponding sensors on the head of the operating person.

Furthermore, the display field can be configured as a display of an operating panel or as a screen, such as an external screen, of a video panel.

The operating unit can have a manually actuatable operating element for the display field, wherein the operating element can be configured to execute or enable a control of a unit of the manipulator when the operating unit is coupled with the manipulator and to execute or enable a control of a unit of the display field when the operating unit is coupled with the display field.

After a coupling between the operating unit and the display field has taken place, a display element, such as, for example, a mouse pointer or the like, can be visible on the display field which can be positioned or respectively controlled on the display field by an operating element of the operating unit. The visible display element can be emphasized on the surface of the display field either as a pointer or as a graphical marking or as an icon, tile, or the like. The movement of the display element on the surface of the display field can take place here by controlling the operating element of the operating unit. This makes it possible, for example, to activate a program by arranging the display element on a symbol displayed on the display field. If, for example, the operating element is configured as a button on the operating unit, the configured program or the correspondingly configured application can be started accordingly by actuating the button. In the case that the operating unit is coupled with the manipulator in the other operating mode, the button on the operating unit can be configured for controlling the manipulator, such as the robotic arm. Moreover, when the operating unit is coupled with the manipulator, a control element of the operating unit can be provided for actuating an end effector, wherein the corresponding control element can be used for controlling the display element on the display field when the operating unit is coupled with the display field.

The display field can be configured as an operating panel for the surgical robot, wherein general functions and settings of the devices, such as, for example, a camera control unit, light source, HF generator, ultrasonic generator, insufflator, video recorder, video router, operation illumination, operating table, etc. used in combination with the surgical robot can be controlled via the operating panel.

In another embodiment, the display field can be configured as a screen or monitor for displaying, for example, an endoscope image or the like, wherein is it possible that a corresponding display element can be displayed on the monitor or screen when the operating unit is coupled with the monitor or respectively screen, wherein the display element can be controlled accordingly with the operating unit or respectively the operating element or by a button on the operating unit. For example, a corresponding menu can be displayed on the monitor or respectively screen after the operating unit has been coupled with the monitor or respectively screen, from which menu corresponding functions can be selected. The operating unit or its operating element can activate or deactivate one or more functions and emphasize, for example, anatomical landmarks or pathologies using automatic image processing. The display of information on the monitor or the screen can be activated or deactivated and the amount of the displayed information which is based accordingly on the selected function can be controlled. Graphically emphasized preferred points can be provided in the displayed image by the operating element or the operating elements, for example, by plotting points or lines. In one embodiment, plotted lines or points can enable corresponding geometric dimensions, such as, for example, lengths or surface areas, to be measured.

Controlling the overlay of, for example pre-operative, image data in the form of sectional images or 3D models can be provided. In doing so, the selection of a corresponding data set, the image plane, the displayed model elements as well as the rotation and the enlargement or the position and the transparency with which the image data are overlaid into the image can be checked.

Furthermore, a surgical navigation system for the surgical robot is used so that, for example, the operating unit or respectively operating elements of the operating unit are coupled with the display field, such as a monitor or screen or operating panel, thereby giving an operating person the capability to select the displayed pre-operative image data, etc. In this case, the selection of a corresponding data set, the displayed model elements, as well as a position and the transparency with which the image data are overlaid into the image can be checked. Reference points on the pre-operative model and on the video image by the operating units can be plotted or selected.

### BRIEF DESCRIPTION OF THE DRAWINGS

Further features of the embodiments will become evident from the description of embodiments, together with the claims and the appended drawings. Embodiments can fulfill individual features or a combination of several features.

The embodiments are described below, without restricting the general idea of the invention, based on exemplary embodiments in reference to the drawings, whereby we expressly refer to the drawings with regard to the disclosure of all details that are not explained in greater detail in the text. In the figures:

FIG. 1 schematically illustrates a surgical robot system in a simplified diagram, and

FIG. 2 schematically illustrates a diagram of a surgical robot system according to another embodiment.

In the drawings, the same or similar elements and/or parts are, in each case, provided with the same reference numerals such that they are not introduced again in each case.

### DETAILED DESCRIPTION

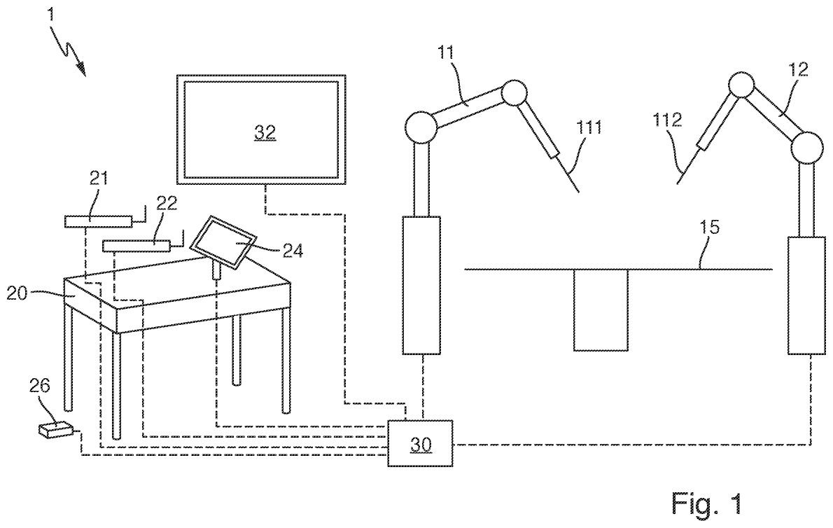

FIG. 1 schematically shows an embodiment of a surgical robot system 1. The surgical robot system 1 has two manipulators (slave devices) 11, 12 for examining and/or treating a person on a table 15, wherein the manipulators 11, 12 can be configured as multi-jointed robot arms.

The manipulators 11, 12 have exchangeable medical instruments 111, 112 on their free ends to examine or operate on persons accordingly. For example, one of the medical instruments 111, 112 can be configured as an endoscope.

The table 15 is provided for the examination of a person so that the person lies on the table 15, for example, for a medical examination of a person. The manipulators 11, 12 can be moved relative to the table 15 with their end-side medical instruments 111 or respectively 112.

Furthermore, the surgical robot system 1 has an operating console 20 with two operating units (master devices) 21, 22 that are manually operable by an operating person. The operating units 21, 22 are connected to a control unit 30 (a controller comprising hardware or a combination of software and hardware) so that, when the operating unit 21 is actuated via the control unit 30, the manipulator 11 connected to the control unit 30 is moved when the operating unit 22 is coupled with the manipulator 12. Furthermore, the second operating unit 21 is connected to the control unit 30 so that, when the operating unit 22 is manually actuated, the manipulator 12 connected to the control unit 30 is actuated when the operating unit 22 is coupled with the manipulator 12.

For example, the operating units 21, 22 are freely movable in all three dimensions so that the manipulators 11, 12 are correspondingly controlled by, for example, an advancement or by a change in angle of the operating units 21, 22.

In addition, the operating console 20 has an operating panel 24 (display), wherein the operating panel 24 has a display field on which a variety of graphical information is displayed, for example in the form of a menu, to display or respectively select corresponding operating modes or the like.

Furthermore, in the exemplary embodiment shown in FIG. 1, a foot button 26 is provided in the region of the operating console 20 and is connected to the control unit 30. When the foot button 26 is actuated, the operating units 21, 22 are each coupled with the manipulators 11, 12 accordingly. In the case of an interruption of the medical examination, the foot button 26, for example, is actuated again by an operating person so that the operating units 21, 22 are decoupled from the manipulators 11, 12 so that, when the operating units 21, 22 are actuated in the decoupled mode, the manipulators 11, 12 are not moved.

The control unit 30 is furthermore connected to a monitor 32 (display) in order to, for example, display the images taken by an endoscope of an examination or operating field for the operating person or the medical personnel.

In addition, the surgical robot system 1 can also be configured with a video processor (comprising hardware or a combination of software and hardware), a light source, an insufflator, a high-frequency surgery device, and/or an ultrasonic surgery device. Furthermore, the surgical system can also be configured with operation lights for illuminating an external operation field, a control for the table 15.

In addition, the surgical robot system 1 can also be configured with a video recorder and/or a video distributor for multiple display screens.

To display and to modify the settings of the surgical robot system, the operating panel 24 is provided, which is embodied, for example, as a touchscreen. In order to change the settings of the surgical robot system, it is possible to control or respectively move a cursor on the surface of the operating panel 24 via another (external) input device, such as, for example, a computer mouse or via a touch pen.

According to this embodiment, it is possible that the operating units 21, 22 are coupled with the operating panel 24 by the actuation of the foot button 26 after a decoupling of the operating units 21, 22 for the manipulators 11, 12, so that, when the decoupling of the operating units 21, 22 from the manipulators 11, 12 takes place and when the coupling of the operating units 21, 22 with the operating panel 24 takes place, for example, by the movement of one of the operating units 21, 22, the cursor or a display element is moved on the operating panel 24.

For example, after the operating units 21, 22 have been decoupled from the manipulators 11, 12 by pressing the foot button 26, one of the operating units 21, 22 can be coupled with the display element, such as, for example, a cursor, on the operating panel 24 when an operating element on one of the operating units 21, 22 is actuated. As a result, it is no longer necessary that, for example, an operating person must manually actuate an external input device, such as, for example, a computer mouse, since now the operating person can actuate the display element on the operating panel 24, for example, via one of the operating units 21, 22. In addition, addi-tional operating elements of the operating units 21, 22 can also be coupled with the control of the operating panel 24 such that it is possible, for example, to select specific operating menus.

For example, the operating units 21, 22 have manually actuatable buttons or sliders or other manually actuatable operating elements in order to control the corresponding graphical display elements or menus when the operating units 21, 22 are coupled with the control for the operating panel 24.

In another embodiment, it can be provided that the foot button 26 is configured such that, in a first operating mode, the foot button 26 couples the operating units 21, 22 with the manipulators 11, 12 at a first pressure point, wherein, at a second pressure point of the foot button 26, the operating units 21, 22 are decoupled from the manipulators 11, 12 and at the same time the operating units 21, 22 are coupled with the control for the operating panel 24. When the foot button 26 is actuated, the operating units 21, 22 are hereby alternatingly coupled with the manipulators 11, 12 or with the operating panel 24.

In another embodiment, it can be provided that, for the coupling of the operating units 21, 22 with the operating panel 24 or respectively the control of the operating panel 24, a sensor device is provided which is configured to detect the gaze of an operating person toward the operating panel 24 in order to couple the operating units 21, 22 with the operating panel 24 or respectively its control when the operating units 21, 22 are decoupled from the manipulators 11, 12 and when a gaze detected by the sensor device is directed at the operating panel 24.

In addition, it can be provided that in another embodiment that, after a decoupling between the operating units 21, 22 and the manipulators 11, 12 has taken place, a speech command is detected by a microphone or the like so that, after a corresponding speech command has been detected, the operating units 21, 22 are coupled with the control of the operating panel 24.

Alternatively, in other embodiments, sensor devices for detecting and evaluating electromyogram signals or electroencephalogram signals of an operating person on the control console 20 control a coupling of the operating units 21, 22 with the control of the operating panel 24.

If one of the operating units 21, 22 is coupled with the control of the operating panel 24, it is possible that an indicator element is visible on the visible field of the operating panel 24, which can be controlled, i.e., positioned on the display field, via an operating element of the operating unit. The indicator element can be embodied, for example, as a pointer, such as, for example, a mouse pointer or the like. Alternatively, the indicator element can be configured as a graphical marking. Depending on the movement of the operating unit or respectively of an operating element of the operating unit, an icon or a tile, for example, on the surface of the operating panel 24 is emphasized.

In addition, a program, the icon of which is displayed on the display field of the operating panel, can be selected by actuating the operating units or respectively one of the operating elements of the operating unit, such as, for example, a button.

General functions and settings of the devices, such as, for example, a camera control unit, light source, HF generator, ultrasonic generator, insufflator, video recorder, video router, operation illumination, control of the operating table, etc. used in combination with the surgical robot system 1 can be controlled via the operating panel 24.

It is also possible in another embodiment that, after the operating units 21, 22 have been decoupled from the manipulators 11, 12, the operating units 21, 22 are connected to the monitor 32 or respectively the control of the monitor 32 (in-stead of to the control for the operating panel 24). Accordingly, the interactions described above with the operating panel 24 by the operating units 21, 22 can also be executed for the interactions with the monitor 32. For example, after at least one of the operating units 21, 22 is coupled with the monitor 32, one or more functions or programs or the like can be selected and/or controlled via a menu on the display of the monitor 32.

Visual diagrams of a detected operation or examination area can be displayed on the monitor 32. For example, after one or more image processing functions or the like are activated, it is possible to emphasize image processing of mark-ings, such as, for example, landmarks or pathologies or the like. In doing so, it is possible to activate and/or deactivate the display of information and to control the amount of displayed information which is based on one or more functions that are provided using image, device, or instrument information.

In addition, it is possible to graphically emphasize selected or specific points of images on the monitor 32, for example, by plotting points or lines, by actuating the operating units 21, 22 coupled with the monitor 32 and, if applicable, by actuating operating elements of the operating units 21, 22. In addition, it is also possible to determine an evaluation using the plotted points or lines, for example, by de-termining geometric dimensions such as length or surface areas.

Furthermore, it is possible in one embodiment to overlay image data, such as, for example, pre-operative image data in the form of sectional images or 3D models, on the monitor 32 and to control them by the operating units 21, 22 coupled with the monitor 32. Here, for example, corresponding data sets or image planes and displayed model elements as well as the rotation or enlargement or the position and the transparency with which the image data are overlaid into the image can be checked by actuating the operating units or operating elements of the operating units 21, 22.

It is also possible in one embodiment that the surgical robot system 1 is used using a surgical navigation system, wherein the displayed pre-operative image data can be selected by coupling the operating elements of the operating units 21, 22 with the monitor 32, configured as a video panel, or alternatively with an operating panel of the surgical navigation system. In this case, the selection of a corresponding data set, the displayed model elements, and the position or the transparency with which the image data are overlaid into the image can be checked, by the operating units 21, 22. In addition, it is also possible, using the operating units 21, 22, to plot or select reference points on a pre-operative model as well as a video image displayed on the monitor 32.

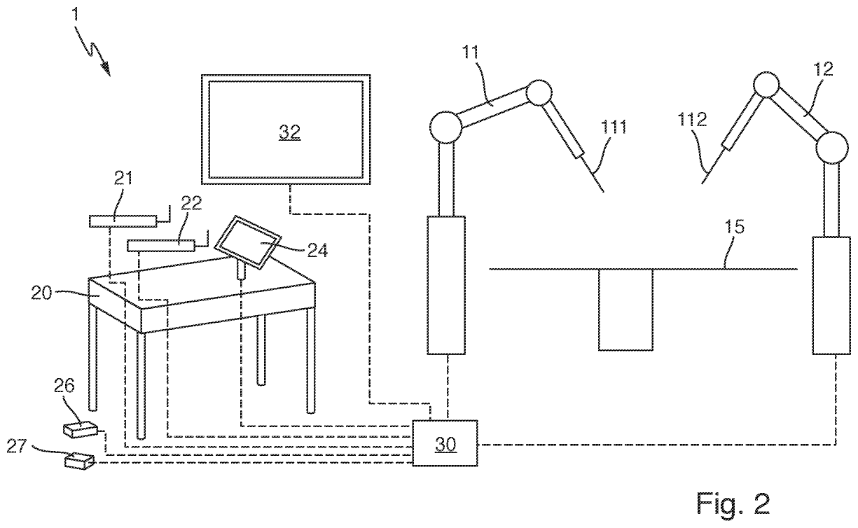

FIG. 2 schematically shows another exemplary embodiment of a surgical robot system 1, wherein, in contrast to the exemplary embodiment shown in FIG. 1, another foot button 27 is provided for coupling the operating units 21, 22 to the operating panel 24, after the manipulators 11, 12 are decoupled, in the region of the operating console 20 in addition to the foot button 26 for coupling/decoupling the operating units 21, 22 with/from the manipulators 11, 12.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE NUMBERS

1 Surgical robot
11 Manipulator
12 Manipulator
15 Table
20 Operating console
21 Operating unit
22 Operating unit
24 Operating panel
26 Foot button
27 Foot button
30 Control unit
32 Monitor
111 Medical instrument
112 Medical instrument

What is claimed is:

1. A surgical robot system comprising:
at least one slave manipulator for handling a surgical instrument within a body of a patient;
a control console, comprising:
at least one master operating unit configured to be manually operated by an operating person to at least control the at least one slave manipulator;
a display for displaying information; and
at least one switch configured to:
couple the at least one master operating unit with the at least one slave manipulator;
decouple the at least one master operating unit from the at least one slave manipulator, and
when the at least one master operating unit is decoupled from the at least one slave manipulator, couple the at least one master operating unit with the display.

2. The surgical robot system according to claim 1, wherein, when the at least one master operating unit is coupled with the display, a display element on the display is activated by actuating the at least one master operating unit.

3. The surgical robot system according to claim 1, wherein the at least one switch is configured as one of a foot pedal or a button.

4. The surgical robot system according to claim 1, wherein the at least one switch is configured such that, at a first switching point, the at least one master operating unit is coupled with the at least one slave manipulator and the at least one master operating unit is decoupled from the display and at a second switching point, the at least one master operating unit is decoupled from the at least one slave manipulator and the at least one master operating unit is coupled with the display.

5. The surgical robot system according to claim 1, wherein, before the at least one switch is actuated, the at least one master operating unit is decoupled from the display and/or during or after actuation of the at least one switch, the at least one master operating unit is decoupled from the at least one manipulator.

6. The surgical robot system according to claim 1, wherein the decoupling and the coupling are one of executed at a same time or executed temporally one after another.

7. The surgical robot system according to claim 1, wherein the at least one switch comprises a gaze sensor, wherein the gaze sensor is configured to couple the at least one master operating unit with the display when the gaze sensor detects that a gaze of the operating person is oriented toward the display.

8. The surgical robot system according to claim 1, wherein the at least one switch comprises a speech recognition device, wherein the speech recognition device is configured to couple the at least one master operating unit with the display when the speech recognition device detects a speech utterance of the operating person.

9. The surgical robot system according to claim 8, wherein the speech utterance is a predetermined speech command.

10. The surgical robot system according to claim 1, wherein the at least one switch comprises an electromyography receiver for receiving electromyography signals of the operating person, wherein the electromyography receiver is configured to couple the at least one master operating unit with the display when the electromyography receiver detects electromyography signals of the operating person.

11. The surgical robot system according to claim 1, at least one switch comprises an electroencephalography receiver for receiving electroencephalography signals of the operating person, wherein the electroencephalography receiver is configured to couple the at least one master operating unit with the display when the electroencephalography receiver detects encephalography signals of the operating person.

12. The surgical robot system according to claim 1, wherein the display is configured as one of an operating panel or as an external, screen of a video panel.

13. The surgical robot system according to claim 2, wherein:
the at least one master operating unit comprises a manually actuatable operating element for the display, and
the manually actuatable operating element is configured to execute or enable a control of a portion of the at least one slave manipulator when the at least one master operating unit is coupled with the at least one slave manipulator and to execute or enable a control of a portion of the display when the at least one master operating unit is coupled with the display.

14. The surgical robot system according to claim 13, wherein the manually actuatable operating element is configured as one of a button or ball.

15. A method for operating a surgical robot system, the method comprising:
coupling at least one master operating unit with the least one slave manipulator;
decoupling the at least one master operating unit from the at least one slave manipulator, and
when the at least one master operating unit is decoupled from the at least one slave manipulator, coupling the at least one master operating unit with a display.

16. The method according to claim 15, wherein, further comprising,
when the at least one master operating unit is coupled with the display, activating a display element on the display by actuating the at least one master operating unit.

17. A control apparatus for controlling surgical robot system, the control apparatus comprising:
a controller comprising hardware, the controller being configured to:
couple at least one master operating unit with at least one slave manipulator;
decouple the at least one master operating unit from the at least one slave manipulator; and
when the at least one master operating unit is decoupled from the at least one slave manipulator, couple the at least one master operating unit with a display.

18. The surgical robot system according to claim 1, wherein, wherein, the controller is further configured to, when the at least one master operating unit is coupled with the display, activate a display element on the display by actuating the at least one master operating unit.
